(19) United States
(12) Patent Application Publication
(10) Pub. No.: US 2018/0368735 A1
(43) Pub. Date: Dec. 27, 2018

(54) Systems and Methods For Assisting Body Movement of A Person With Impaired Sensation

(71) Applicant: Jonathan Campbell, Herndon, VA (US)

(72) Inventor: Jonathan Campbell, Herndon, VA (US)

(21) Appl. No.: 16/014,388

(22) Filed: Jun. 21, 2018

Related U.S. Application Data
(60) Provisional application No. 62/523,346, filed on Jun. 22, 2017.

(51) Int. Cl. A61B 5/11; G08B 21/22; A61B 5/00; A61B 5/103

(52) U.S. Cl. CPC A61B 5/1115; G08B 21/22; A61B 5/6894; A61B 5/1036; A61B 5/6892; A61B 5/1114; A61B 5/4023; A61B 2562/0247; A61B 5/6891

(57) ABSTRACT

Systems and methods for assisting body movement of a user are disclosed according to various embodiments. In one example, a disclosed system includes: at least one sensor, a feedback device, a memory storing pre-configured values, and a processor. The at least one sensor is configured for measuring force or pressure applied by the user on a seat. The feedback device is configured for providing feedback to the user based on a force or pressure measurement from the at least one sensor. The processor is configured to: receive the force or pressure measured by the at least one sensor, calculate a percentage based on the force or pressure measurement and the pre-configured values stored in the memory, determine whether the calculated percentage falls within a predetermined range, and cause the feedback device to provide a feedback to the user when the percentage falls within the predetermined range. The feedback indicates the force or pressure applied by the user on the seat.

### CROSS-REFERENCE TO RELATED APPLICATION

This application claims priority to U.S. Provisional Application No. 62/523,346 filed on Jun. 22, 2017, the entirety of which is hereby incorporated by reference herein.

### FIELD

The present disclosure generally relates to systems and methods for assisting body movement of a person with impaired sensation. More specifically, some embodiments of the present disclosure are directed to systems and methods for measuring and monitoring pressure or angle applied by a user on a seat and for providing real-time feedback regarding the monitored parameters to the user.

### BACKGROUND

Impaired sensation, due to injury, disease, or genetic condition is a common complication among elderly, diabetic and spinal cord injured (“SCI”) populations. Impaired sensation in affected areas can lead to a patient failing to recognize warning signs such as pain and discomfort on areas of the skin that have pressure applied. These areas often include, but are not limited to, bony prominences such as the heel, the coccyx, and ischial tuberosities (the “sit bones”).

For wheelchair-bound individuals with impaired sensation, the process of transferring or moving from one seat to another (e.g. from a wheelchair to a couch) can be especially difficult. Lack of sensation makes it difficult for such individuals to recognize when his or her backside has lifted from the seat from which the transfer is to occur. Lack of sensation also makes it difficult for such individual to determine how much weight his or her legs are bearing during the transfer process.

The most efficient way to stand from a seated position is by leaning forward. However, leaning forward puts persons with impaired sensation at increased risk of falling due to difficulties that such persons have when determining balance and position during movement. In order to transfer successfully and safely, a person with impaired sensation must rely on muscle memory and equilibrium to maintain balance and gauge how far out of the seat he or she is in both vertical and horizontal planes.

### SUMMARY

The present disclosure generally relates to systems and methods for assisting body movement of a person with impaired sensation. More specifically, some embodiments of the present disclosure are directed to systems and methods for measuring and monitoring pressure or angle applied by a user on a seat and for providing real-time feedback regarding the monitored parameters to the user.

The exemplary embodiments disclosed herein are directed to solving the issues relating to one or more of the problems presented in the prior art, as well as providing additional features that will become readily apparent by reference to the following detailed description when taken in conjunction with the accompanying drawings. In accordance with various embodiments, exemplary systems, methods, devices and computer program products are disclosed herein. It is understood, however, that these embodiments are presented by way of example and not limitation, and it will be apparent to those of ordinary skill in the art who read the present disclosure that various modifications to the disclosed embodiments can be made while remaining within the scope of the present disclosure.

In one embodiment, a system is disclosed for assisting body movement of a user. The system includes: at least one sensor, a feedback device, a memory storing pre-configured values, and a processor. The at least one sensor is configured for measuring force or pressure applied by the user on a seat. The feedback device is configured for providing feedback to the user based on a force or pressure measurement from the at least one sensor. The processor is configured to: receive the force or pressure measurement measured by the at least one sensor, calculate a percentage based on the force or pressure measurement and the pre-configured values stored in the memory, determine whether the calculated percentage falls within a predetermined range, and cause the feedback device to provide a feedback to the user when the percentage falls within the predetermined range. The feedback indicates the force or pressure applied by the user on the seat.

In another embodiment, a system is disclosed for assisting body movement of a user. The system includes: at least one sensor, a feedback device, a memory storing pre-configured values, and a processor. The at least one sensor is coupled to a thigh of the user and configured for measuring an angle between the thigh of the user and a ground surface under a seat of the user. The feedback device is configured for providing feedback to the user based on an angle measurement from the at least one sensor. The processor is configured to: receive the angle measurement measured by the at least one sensor, calculate a percentage based on the angle measurement and the pre-configured values stored in the memory, determine whether the calculated percentage falls within a predetermined range, and cause the feedback device to provide a feedback to the user when the percentage falls within the predetermined range. The feedback indicates the angle between the thigh of the user and the ground surface.

In yet another embodiment, a method for assisting body movement of a user is disclosed. The method includes: with at least one sensor, measuring force or pressure applied by a user on a seat; calculating a percentage based on the force or pressure and a pre-configured value stored in a memory; determining whether the percentage falls within a predetermined range; and providing a feedback to the user when the percentage falls within the predetermined range. The feedback indicates the force or pressure applied by the user on the seat.

In still another embodiment, a system is disclosed for assisting body movement of a user. The system includes: a cushion for a seat, at least one sensor, a feedback device, a memory storing pre-configured values, and a processor. The at least one sensor is coupled to the cushion and configured for measuring force or pressure applied by the user on the seat. The feedback device is configured for providing feedback to the user based on a force or pressure measurement from the at least one sensor. The processor is configured to: receive the force or pressure measurement measured by the at least one sensor, calculate a percentage based on the force or pressure measurement and the pre-configured values stored in the memory, determine whether the calculated percentage falls within a predetermined range, and cause the feedback device to provide a feedback to the user when the percentage falls within the predetermined range. The feedback indicates the force or pressure applied by the user on the seat.

### BRIEF DESCRIPTION OF THE DRAWINGS

Various exemplary embodiments of the present disclosure are described in detail below with reference to the following Figures. The drawings are provided for purposes of illustration only and merely depict exemplary embodiments of the present disclosure to facilitate the reader's understanding of the present disclosure. Therefore, the drawings should not be considered limiting of the breadth, scope, or applicability of the present disclosure. It should be noted that for clarity and ease of illustration these drawings are not necessarily drawn to scale.

FIG. 1 is an exploded and perspective view of a pressure monitoring system, in accordance with some embodiments of the present disclosure.

FIG. 2 is a collapsed view of the system of FIG. 1, in accordance with some embodiments of the present disclosure.

FIG. 3 is a system diagram of operable connections of the components of a pressure monitoring system, in accordance with some embodiments of the present disclosure.

FIG. 4 is a system diagram of operable connections of the components of a pressure monitoring system, in accordance with some embodiments of the present disclosure.

FIG. 5 is a flow diagram illustrating operations of a pressure monitoring system, in accordance with some embodiments of the present disclosure.

FIG. 6 is a side view of a user angle monitoring system, in accordance with some embodiments of the present disclosure.

FIG. 7 is a side view of the user angle monitoring system in FIG. 6 during its operation, in accordance with some embodiments of the present disclosure.

FIG. 8 is a system diagram of operable connections of the components of a user angle monitoring system, in accordance with some embodiments of the present disclosure.

FIG. 9 is a system diagram of operable connections of the components of a user angle monitoring system, in accordance with some embodiments of the present disclosure.

FIG. 10 is a flow diagram illustrating operations of a user angle monitoring system, in accordance with some embodiments of the present disclosure.

### DETAILED DESCRIPTION

Various exemplary embodiments of the present disclosure are described below with reference to the accompanying figures to enable a person of ordinary skill in the art to make and use the present disclosure. As would be apparent to those of ordinary skill in the art, after reading the present disclosure, various changes or modifications to the examples described herein can be made without departing from the scope of the present disclosure. Thus, the present disclosure is not limited to the exemplary embodiments and applications described and illustrated herein. Additionally, the specific order and/or hierarchy of steps in the methods disclosed herein are merely exemplary approaches. Based upon design preferences, the specific order or hierarchy of steps of the disclosed methods or processes can be re-arranged while remaining within the scope of the present disclosure. Thus, those of ordinary skill in the art will understand that the methods and techniques disclosed herein present various steps or acts in a sample order, and the present disclosure is not limited to the specific order or hierarchy presented unless expressly stated otherwise.

This description of the exemplary embodiments is intended to be read in connection with the accompanying drawings, which are to be considered part of the entire written description. In the description, relative terms such as “lower,” “upper,” “horizontal,” “vertical,”, “above,” “below,” “up,” “down,” “top” and “bottom” as well as derivative thereof (e.g., “horizontally,” “vertically,” “downwardly,” “upwardly,” etc.) should be construed to refer to the orientation as then described or as shown in the drawing under discussion. These relative terms are for convenience of description and do not require that the apparatus be constructed or operated in a particular orientation.

Various embodiments provide methods and systems for assisting body movement of a user who may have an impaired sensation. In some embodiments, the system can measure and monitor an amount of pressure a user is applying to a seat and provide feedback to the user during transfer of the user's body from the seat to another. For example, the system may comprise a pressure-sensitive mat and a monitoring or feedback device. The pressure-sensitive mat may be placed between a seat cushion and a chair. When a user sits on the cushion, the system will monitor the pressure applied by the user to the pressure-sensitive mat and will notify the user with an audible alarm or other means as the pressure changes. For example, as a user begins to lift his or her body out of the seat, an audible tone will sound, alerting the user as the pressure on the seat changes.

In some embodiments, other feedback could be used, such as an LED or light that changes as the pressure changes. In some embodiments, haptic feedback is provided, e.g. by a vibrating smart watch that vibrates when the pressure changes. In other embodiments, one or more of audible, visual, and haptic feedbacks may be provided to the user as the pressure changes. In still other embodiments, the pitch or frequency of the audible tone will vary according to the amount of pressure applied to the pressure-sensitive mat. In embodiments where an LED or haptic feedback is used, the color or brightness of the LED, or the strength, frequency, or pattern of haptic feedback will vary based on the amount of the pressure applied.

In certain embodiments, the system will be calibrated according to a pressure applied to the pressure-sensitive mat while the seat is unoccupied (referred to as the “Empty Seat Pressure”) and according to the user's weight as applied to the pressure-sensitive mat when seated (referred to as the “Seated Pressure”). A user may adjust pre-configured values that will determine a point at which the feedback occurs during a transfer. For example, a user may adjust an “Upper Threshold” of the system to 90% of the user's Seated Pressure. As the pressure applied by the user drops below that threshold, the system will provide user feedback, which continues until the pressure applied drops below a “Lower Threshold” that is for example 10% of the user's Seated Pressure. A user may not want to set the Lower Threshold to 0%, as one would not want the alarm to continue while the seat is empty.

In other embodiments, the system comprises an accelerometer and a monitoring or feedback device. Rather than measuring pressure, an accelerometer, which may be placed on the body (for example, on the thigh) of the user, measures the angular displacement of that part of the body. In this case, the system provides user feedback according to changes in the angular displacement of the body, in order to assist the user in determining how far he or she has lifted out of his or her seat. Auditory, visual and/or haptic feedback will change according to the angular displacement measured by the accelerometer.

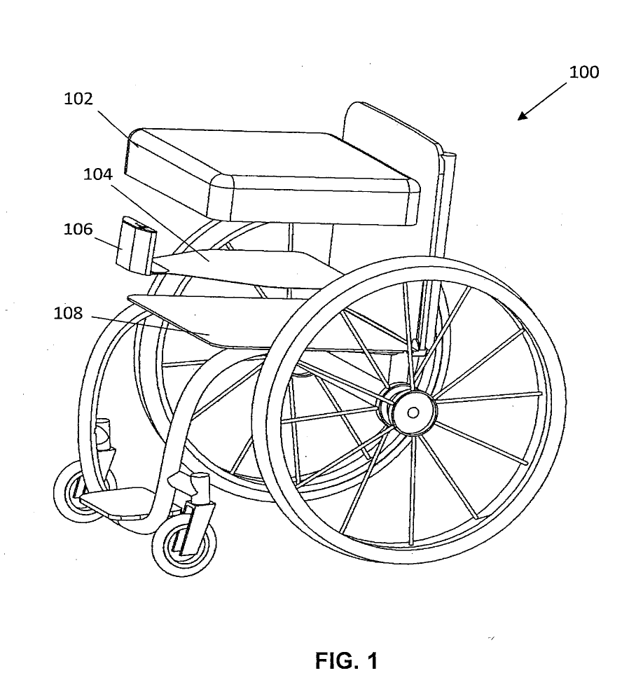

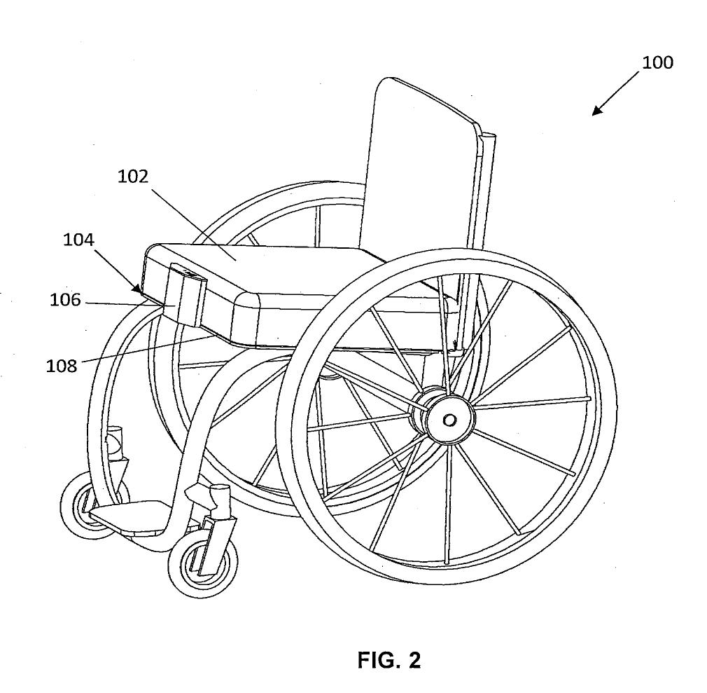

An illustrative embodiment of a wheelchair-based pressure monitoring system 100 is shown in FIGS. 1 and 2. While the example shown in FIGS. 1 and 2 involves a wheelchair, other examples may use other types of equipment and do not require a wheelchair. System 100 includes a wheelchair seat cushion 102, a sensor mat 104, a microprocessor unit 106, and a wheelchair seat base 108. FIG. 1 illustrates an exploded and perspective view of these elements, and FIG. 2 shows these elements in their normal positions. The microprocessor unit 106 is operably coupled to the sensor mat 104, which is placed between the wheelchair seat cushion 102 and the wheelchair seat base 108.

In some embodiments, the sensor mat 104 comprises a pressure sensitive mat embedded with one or more piezo-resistive force sensors. When a user sits down on the chair of system 100, the user's weight is detected and measured as a force by the sensor(s) which generate(s) a signal, proportional to the force, which is sent to the microprocessor unit 106. The system may be calibrated to convert the signals from these sensors into a pressure placed on the skin of the system user. Pressure and force may be used interchangeably throughout this disclosure in the context of sensors, as pressure is force per unit area. The force sensor(s) of sensor mat 104 may be operably connected to generate a single, combined output signal from the mat 104 to the microprocessor unit 106. In some embodiments, the sensor mat 104 comprises a plurality of sensors, with each sensors measuring a defined area of the sensor mat 104, which outputs a discrete signal for each sensor. This allows the system to measure not only an average pressure or force on a user's skin but the particular pressure or force on any pressure point or particular location. The defined areas may also comprise the left and right, front and back, or other regions of the chair.

While the sensor mat 104 is shown in FIGS. 1 and 2 as a separate component, the sensor mat 104 may be integrated with the wheelchair seat cushion 102 and/or the base 108. Furthermore, the sensor mat 104 may be used in other applications. For instance, in some embodiments the mat 104 is integrated on the arm or foot rests or back support or other additional areas of wheelchair, on the contact point of a crutch, in a bed, or in the clothing of a person at risk of pressure problem areas. For example, sensors or a sensor mat 104 could be placed in the heels of a patient's shoes. These additional sensors may be connected via wires or wirelessly to a microprocessor unit. In these embodiments, the system may monitor every connected sensor and provide notifications to the patient.

While shown in FIGS. 1 and 2 as a unitary device positioned adjacent to the sensor mat 104, the microprocessor unit 106 may comprise a plurality of separate components located elsewhere in the system. The microprocessor unit 106 may comprise a microprocessor, dip switches, a mobile device or other programming or user interface, internal memory, indicator LEDs or other display device, speaker, vibrating or other physical indicator and a power source. The power source may be, for example, a rechargeable lithium ion battery dedicated to powering the system 100, or it may be the power source of a powered wheelchair, or power from another source such as an electrical outlet on a wall.

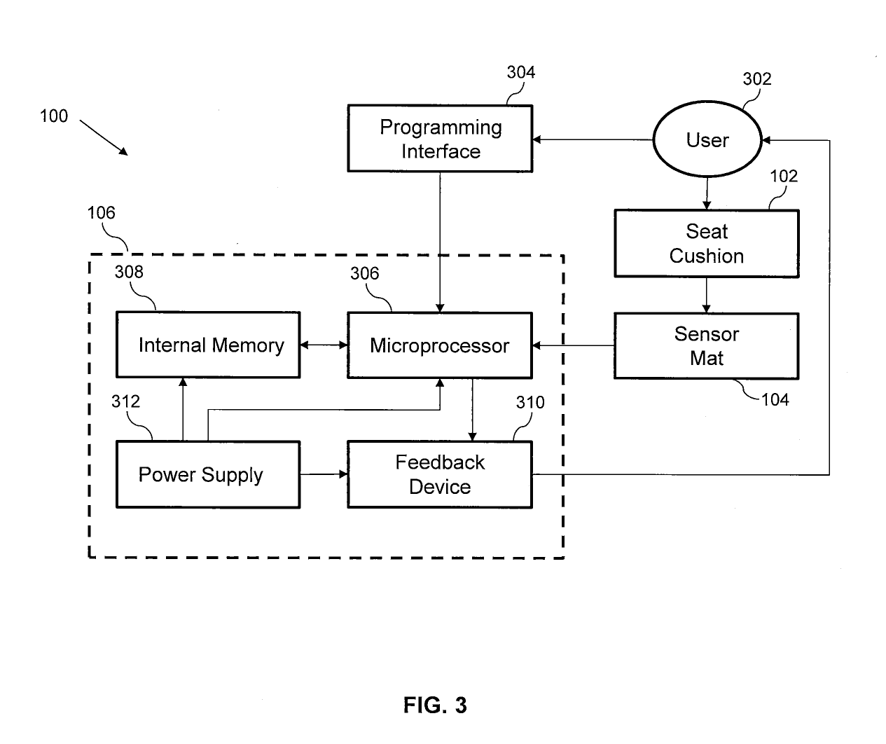

A system diagram of the operable connections between the components of a pressure monitoring system, including a detailed diagram of components of a microprocessor unit 106, is shown in FIG. 3. The microprocessor unit 106 in this example includes a microprocessor 306, an internal memory 308 operatively coupled to the microprocessor 306, a feedback device 310 operatively coupled to the microprocessor 306, and a power supply 312 that provides power to the microprocessor 306, the internal memory 308, and the feedback device 310. In various embodiments, the microprocessor unit 106 may be mounted on any seat a user with impaired sensation could sit on.

The sensor mat 104 is configured for measuring force or pressure applied by the user 302 on a seat, e.g. the wheelchair as shown in FIG. 1. For example, the user 302 sits on the wheelchair seat cushion 102, thereby applying sitting pressure. In turn, the user's seat cushion 102 applies pressure to the sensor mat 104 which may be embedded with piezoelectric force sensors or other force or pressure sensors.

The user 302 may enter system pre-configured or pre-set values/thresholds and/or other parameters using a programming or user interface 304, such as a keypad, handheld device or a computer. The user 302 can select from any combination of settings, specifically the “Upper Threshold”, “Lower Threshold”, and type of feedback (i.e., visual, auditory, haptic, or some combination of these). In addition, the pre-configured values may comprise at least one of: a full seat value representing a force or pressure measured when the user fully sits on the seat; an empty seat value representing a force or pressure measured when the seat is not occupied by the user; the upper threshold; and the lower threshold. The user's presets or the system's pre-configured values are stored on internal memory or flash drive 308 by the microprocessor 306. The microprocessor 306 receives the user's presets from the programming interface 304 and stores it in internal memory 308 in addition to receiving pressure signals from the sensor mat 104. In one embodiment, a user with impaired sensation may need an assistant, e.g. a therapist or a nurse, to help to determine these pre-configured values based on a trial of body movement from and/or onto the seat with the feedback system. In another embodiment, a user may adjust or modify these pre-configured values after the user changes weight and/or shape of his or her body.

The microprocessor 306 may perform a calibration of the system 100. For example, the microprocessor 306 may calibrate the empty seat value to zero; calibrate the full seat value to generate a calibrated full seat value in accordance with the calibration of the empty seat value; and calibrate the force or pressure measured by the sensor mat 104 to generate a calibrated value in accordance with the calibration of the empty seat value. The microprocessor 306 can calculate a percentage based on a ratio between the calibrated value and the calibrated full seat value, and determine whether the percentage falls into a predetermined range between the lower threshold and the upper threshold. In one embodiment, the upper threshold represents a first percentage of the calibrated full seat value; and the lower threshold represents a second percentage of the calibrated full seat value. The second percentage (e.g. 10% or 15%) is smaller than the first percentage (e.g. 85% or 90%).

The microprocessor 306 receives signals from the sensor mat 104 when pressure is applied (e.g., from the weight of a user). As discussed above, the system calibrates the “Empty Seat Pressure” by measuring the amount of pressure applied to the sensor mat 104 when the user is not seated; calibrates the “Seated Pressure” by measuring the amount of pressure applied to the sensor mat 104 while the user is seated; and calculates a “Pressure Percentage” that reflects the percentage of the Seated Pressure that is being applied to the sensor mat 104 at any specific point in time. The sensor mat 104 can continuously monitor force or pressure applied by the user 302 on the seat, and send the monitored force or pressure to the microprocessor 306. Based on the monitored force or pressure from the sensor mat 104, the microprocessor 306 can monitor a change of the pressure percentage.

The microprocessor 306 may determine whether the pressure percentage falls into a predetermined range between the lower threshold and the upper threshold. If so, the microprocessor 306 may instruct the feedback device 310 to provide user feedback to the user 302, where the feedback will change (by strength, frequency, color, volume, etc.) according to changes of the pressure percentage. The feedback indicates the force or pressure applied by the user 302 on the seat. As such, the user 302 can receive real-time feedback while he or she moves the body from or to the seat. This is especially beneficial to a user with impaired sensation.

While the microprocessor 306 monitors a change of the pressure percentage, the microprocessor 306 can determine whether the user 302 intends to lift from the seat or sit onto the seat. In one example, the microprocessor 306 detects that the pressure percentage changes from above the upper threshold to below the upper threshold. As such, the microprocessor 306 determines that the user begins to lift from the seat. Then, the microprocessor 306 may cause the feedback device 310 to start providing the feedback to the user; compare the pressure percentage with the lower threshold; and cause the feedback device 310 to stop providing the feedback to the user in the event the pressure percentage is smaller than the lower threshold. In another example, the microprocessor 306 detects that the pressure percentage changes from below the lower threshold to above the lower threshold. As such, the microprocessor 306 determines that the user begins to sit onto the seat. Then, the microprocessor 306 may cause the feedback device 310 to start providing the feedback to the user; compare the pressure percentage with the upper threshold; and cause the feedback device 310 to stop providing the feedback to the user in the event the pressure percentage is greater than the upper threshold.

The feedback provided to the user 302 may be in any format of: a visual feedback, an auditory feedback, a haptic feedback, or combinations thereof. In some embodiments, the volume, pitch or frequency of an audible tone of an auditory feedback will vary depending on the value of the pressure percentage. As a non-limiting example, as the pressure percentage falls from 90% to 10%, the pitch of the audible tone will decrease from 600 Hz to 200 Hz. In some embodiments, the color or brightness of an LED feedback will vary depending on the value of the pressure percentage. For example, the pressure percentage may be displayed as a progress bar of LEDs. The progress bar may represent pressure percentage values from 0% to 100%. In some embodiments, the pattern, intensity, and frequency of haptic feedback, will vary depending on the value of the pressure percentage.

In some embodiments, the microprocessor unit 106 may include an optional accessory port (not shown in FIG. 3) that allows for other devices to be connected. These other devices may include, but are not limited to: alternative notification devices, such as a vibrating motor, amplified audible alarms, additional LEDs, or LCD monitor; data collection or logging devices; wireless transmitters that would send alarm notifications to a nurse or caregiver; or other devices.

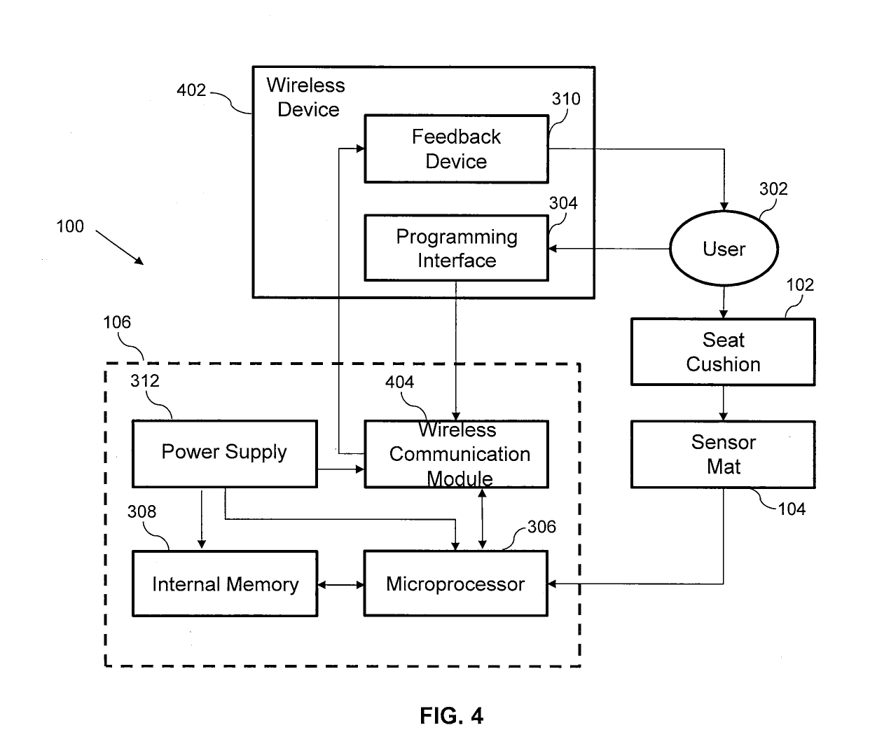

A system diagram of the operable connections between the components of a pressure monitoring system in accordance with some embodiments is shown in FIG. 4. This embodiment differs from that presented in FIG. 3 by the placement of the feedback device 310 into a wireless device 402 together with, optionally, the programming interface 304, and the introduction of the wireless communications module 404 in the microprocessor unit 106. In some embodiments, the wireless device 402 may be a smart phone or smart watch used to receive user inputs from the user 302 and provide detailed feedback signals (in forms of audible, visual, and/or haptic feedbacks) to the user 302. For example, an iPhone®, Apple Watch® or Android Smartwatch® may be used. The user 302 may also view data, including, but not limited to, current pressure values, historical values and statistics and battery levels. The user 302 may make adjustments including, but not limited to, alarm settings, volume settings, feedback types, and all pre-configured values stored in the internal memory 308. The user may also receive feedback from the system via the wireless device 402, such as feedback representing a pressure percentage based on the force or pressure applied by the user 302 on the seat in real-time. In the embodiment shown in FIG. 4, the feedback device 310 and the user interface 304 are operatively coupled to each other and located at or in the wireless device 402. In some embodiments, the microprocessor 306 and the internal memory 308 may also be operatively coupled to or located at or in the wireless device 402.

The wireless communication module 404 enables wireless communication between the wireless device 402 and the microprocessor 306. The microprocessor 306 receives the user's presets from the wireless communication module 404 and stores it in the internal memory 308, and receives pressure signals from the sensor mat 104 with electronic pressure sensor. After calculation of the pressure percentage and comparison with thresholds, feedback signals are communicated back to the wireless device 402 via the wireless communication module 404.

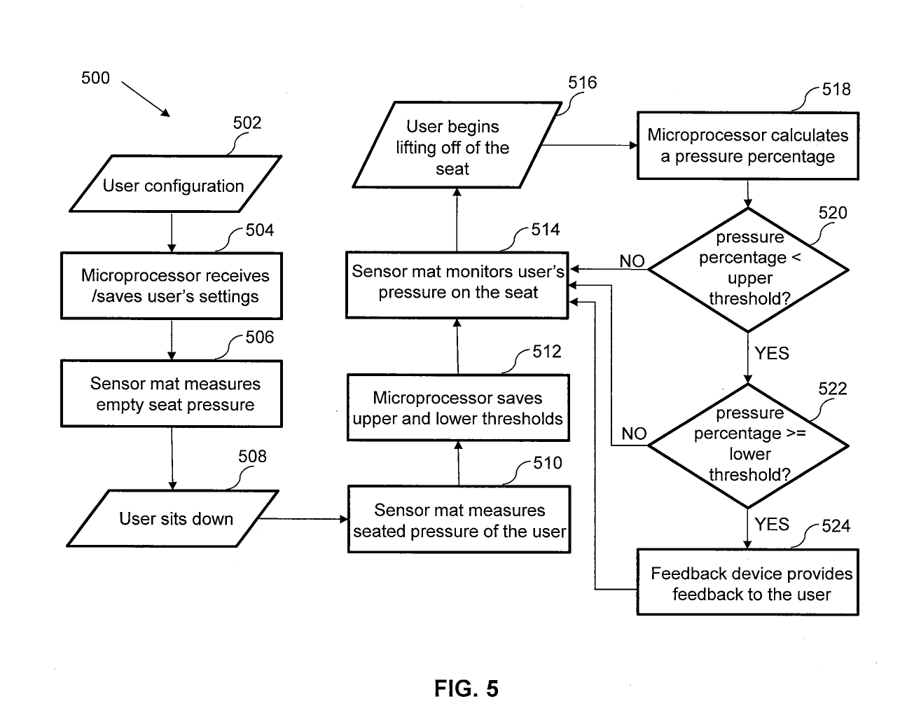

FIG. 5 is a flow diagram illustrating operations 500 of a pressure monitoring system, e.g. the pressure monitoring system 100 in FIGS. 1-4, in accordance with some embodiments of the present disclosure. At block 502, the user chooses the system settings using the programming interface 304. The user may choose from settings such as: feedback method, which allows the user to choose the type of feedback he or she will receive, e.g. visual, auditory, haptic, or combinations thereof; feedback controls, which allows the user to control feedback options such as volume, brightness, and/or haptic type; and threshold adjustments, which allow the user to set the pressure thresholds. For example, the “Upper Threshold”, e.g. at 90% of the user's full weight, represents a Pressure Percentage at which the system begins to provide user feedback when the user lifts from the seat; the “Lower Threshold”, e.g. at 10% of the user's full weight, is a point where the feedback turns off after the user leaves the seat.

At block 504, the settings are received by the microprocessor 306 and stored in the internal memory 308. At block 506, the sensor mat 104, which comprises a pressure sensitive mat embedded with, for example, one or more piezo-resistive force sensors, measures the “Empty Seat Pressure” when the chair is not occupied by the user. This value is calibrated to represent 0% pressure percentage. At block 508, the user sits down on the seat. At block 510, the pressure-sensitive mat measures the “Seated Pressure” applied by the user upon sitting. This value is calibrated to represent a 100% pressure percentage. The microprocessor stores the upper and lower threshold values into the internal memory 308 at block 512. At block 514, the pressure-sensitive mat begins monitoring any changes in pressure applied by the user on the seat. The user begins to lift from his or her seat at block 516.

The microprocessor calculates a pressure percentage at block 518, e.g. by reading the value from the pressure-sensitive mat and dividing it by the “Seated Pressure” to calculate the “Pressure Percentage.” At block 520, it is determined whether the Pressure Percentage is smaller than the “Upper Threshold.” If so, the process goes to block 522; otherwise, the process returns to block 514 to continue monitoring the user's pressure on the seat.

At block 522, it is determined whether the Pressure Percentage is larger than or equal to the “Lower Threshold.” If so, the process goes to block 524; otherwise, the process returns to block 514 to continue monitoring the user's pressure on the seat. At block 524, the feedback device provides auditory, visual, or haptic feedback to the user. The process continues to block 514 to monitor the user's pressure on the seat. That is, the feedback is continuously provided until the pressure percentage falls outside the range between the lower threshold and the upper threshold.

In some embodiments, a sensor, e.g. an accelerometer, may be placed on the body (for example, on the thigh) of a user and will measure changes in angular displacement to determine how far the user has lifted out of his or her seat. An attribute of an auditory, visual, haptic, or some combination thereof, feedback would change according to the angular displacement measured by the accelerometer.

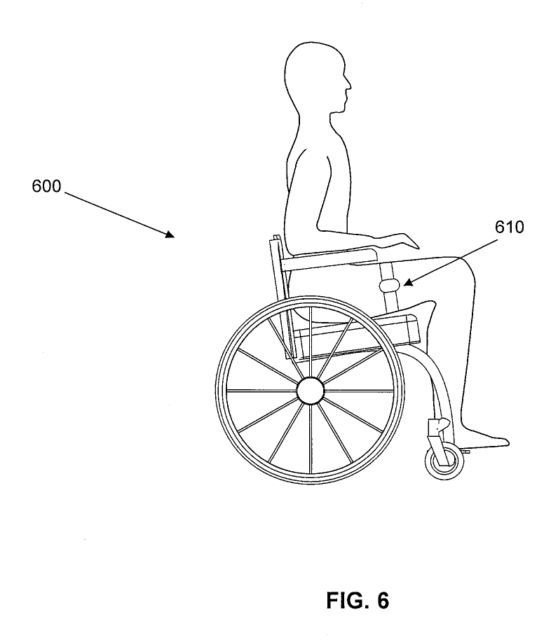

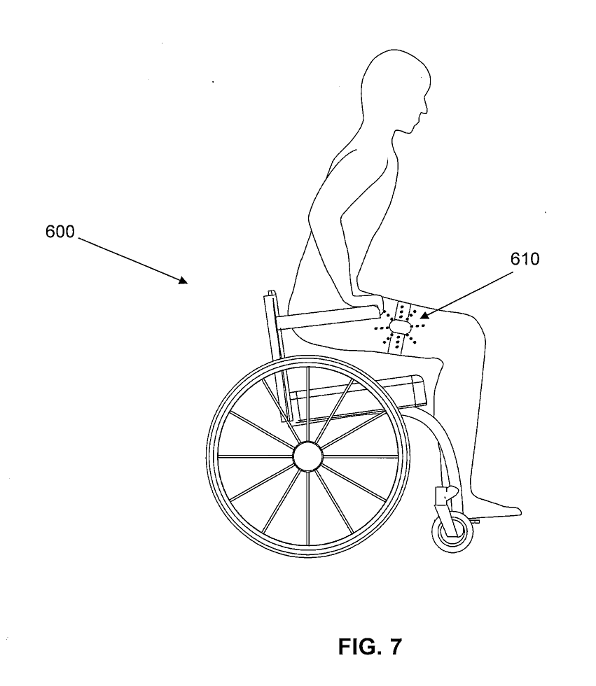

An illustrative embodiment of a user angle monitoring system 600 is shown in FIGS. 6 and 7. FIG. 6 is a side view of the user angle monitoring system 600 when the user sits fully on the seat; while FIG. 7 is a side view of the user angle monitoring system 600 during its operation when the user moves the body from or to the seat.

System 600 includes a sensor unit 610. The sensor unit 610 in this example is attached to a thigh of the user to measure the angle of the user to calculate his or her position relative to the seat he or she was sitting in. The angle may be referred to as user angle and defined as an angle between the thigh of the user and a ground surface under the seat, or as an angle between the thigh of the user and a mat surface of the seat.

The sensor unit 610 may comprise a microprocessor, an accelerometer and/or other sensors, dip switches, a mobile device or other programming or user interface, internal memory, indicator LEDs or other display device, speaker, vibrating or other physical indicator and a power source. The power source may be, for example, a rechargeable lithium ion battery dedicated to powering the system 600, or it may be the power source of a powered wheelchair, or power from another source such as an electrical outlet on a wall. When a user sits down on the chair of system 600, the user's weight is detected and the user angle is measured as angle or percentage (as discussed below) by the sensor(s) which generate(s) a signal sent to the microprocessor. As shown in FIG. 7, when the user lifts from his or her seat, feedback will be provided according to the measured user angle.

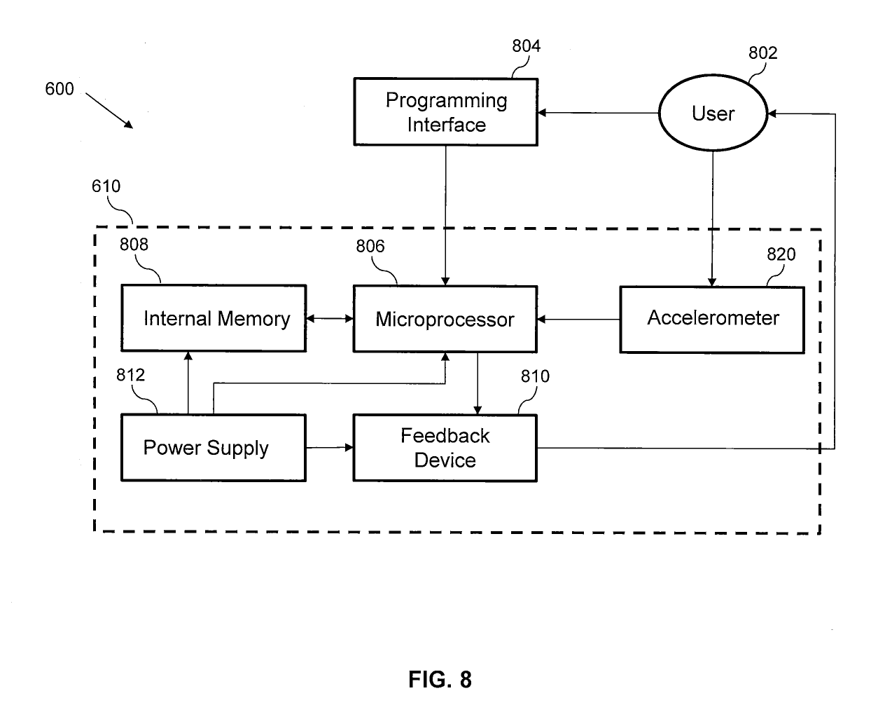

An exemplary system diagram of the operable connections between the components of a user angle monitoring system, including a diagram of components of a sensor unit 610, is shown in FIG. 8. The sensor unit 610 in this example includes a microprocessor 806, an internal memory 808 operatively coupled to the microprocessor 806, a feedback device 810 operatively coupled to the microprocessor 806, an accelerometer 820 operatively coupled to the microprocessor 806, and a power supply 812 that provides power to the microprocessor 806, the internal memory 808, and the feedback device 810.

The accelerometer 820, in this example, is configured for measuring a user angle between the thigh of the user 802 and a ground surface or mat surface. The accelerometer 820 may or may not receive power from the power supply 812. In one example, the accelerometer 820 is inside the sensor unit 610, while in another example, the accelerometer 820 is separate from the sensor unit 610.

The user 802 may enter system pre-configured or pre-set values/thresholds and other parameters using a programming or user interface 804, such as a keypad, handheld device or a computer. The user 802 can select from any combination of settings, specifically the “Upper Threshold”, “Lower Threshold”, and type of feedback (i.e., visual, auditory, haptic, or combinations thereof). In addition, the pre-configured values may comprise at least one of: a full seat value representing a user angle measured when the user fully sits on the seat; an empty seat value representing a user angle measured when a force or pressure applied by the user on the seat reaches a value at which the seat is not occupied by the user; the upper threshold; and the lower threshold. The user's presets or the system's pre-configured values are stored on internal memory or flash drive 808 by the microprocessor 806. The microprocessor 806 receives the user's presets from the programming interface 804 and stores it in internal memory 808 in addition to receiving angle signals from the accelerometer 820.

The microprocessor 806 may perform a calibration of the system 600. For example, the microprocessor 806 may calibrate the empty seat value to zero; calibrate the full seat value to generate a calibrated full seat value in accordance with the calibration of the empty seat value; and calibrate the user angle measured by the accelerometer 820 to generate a calibrated value in accordance with the calibration of the empty seat value. The microprocessor 806 can calculate a percentage based on a ratio between the calibrated value and the calibrated full seat value, and determine whether the percentage falls into a predetermined range between the lower threshold and the upper threshold. In one embodiment, the upper threshold represents a first percentage of the calibrated full seat value; and the lower threshold represents a second percentage of the calibrated full seat value. The second percentage (e.g. 10% or 15%) is smaller than the first percentage (e.g. 85% or 90%).

The microprocessor 806 receives signals from the accelerometer 820 when pressure is applied (e.g., from the weight of a user) and/or a user angle is formed. As discussed above, the system calibrates the “Empty Seat Angle” by measuring the user angle when the user is not seated or when a force or pressure applied by the user on the seat reaches a value at which the seat is not occupied by the user; calibrates the “Seated Angle” by measuring the user angle while the user is seated; and calculates an “Angle Percentage” that reflects the percentage of the Seated Angle that is formed at any specific point in time. The accelerometer 820 can continuously monitor the user angle of the user 802 on the seat, and send the monitored user angle to the microprocessor 806. Based on the monitored user angle from the accelerometer 820, the microprocessor 806 can monitor a change of the angle percentage.

The microprocessor 806 may determine whether the angle percentage falls into a predetermined range between the lower threshold and the upper threshold. If so, the microprocessor 806 may instruct the feedback device 810 to provide user feedback to the user 802, where the feedback will change (by strength, frequency, color, volume, etc.) according to changes of the angle percentage. In this example, the feedback indicates the user angle between the thigh of the user 802 and a ground surface or mat surface of the seat. As such, the user 802 can receive real-time feedback while he or she moves the body from or to the seat. This is especially beneficial to a user with impaired sensation.

While the microprocessor 806 monitors a change of the angle percentage, the microprocessor 806 can determine whether the user 802 intends to lift from the seat or sit onto the seat. In one example, the microprocessor 806 detects that the angle percentage changes from above the upper threshold to below the upper threshold. As such, the microprocessor 806 determines that the user begins to lift from the seat. Then, the microprocessor 806 may cause the feedback device 810 to start providing the feedback to the user; compare the angle percentage with the lower threshold; and cause the feedback device 810 to stop providing the feedback to the user in the event the angle percentage is smaller than the lower threshold. In another example, the microprocessor 806 detects that the angle percentage changes from below the lower threshold to above the lower threshold. As such, the microprocessor 806 determines that the user begins to sit onto the seat. Then, the microprocessor 806 may cause the feedback device 810 to start providing the feedback to the user; compare the angle percentage with the upper threshold; and cause the feedback device 810 to stop providing the feedback to the user in the event the angle percentage is greater than the upper threshold.

The feedback provided to the user 802 may be in any format of: a visual feedback, an auditory feedback, a haptic feedback, or combinations thereof. In some embodiments, the volume, pitch or frequency of an audible tone of an auditory feedback will vary depending on the value of the angle percentage. In a non-limiting example, as the angle percentage falls from 90% to 10%, the pitch of the audible tone will decrease from 600 Hz to 200 Hz. In some embodiments, the color or brightness of an LED feedback will vary depending on the value of the angle percentage. For example, the angle percentage may be displayed as a progress bar of LEDs. The progress bar will represent angle percentage values from 0% to 100%. In some embodiments, the pattern, intensity, and frequency of haptic feedback, will vary depending on the value of the angle percentage.

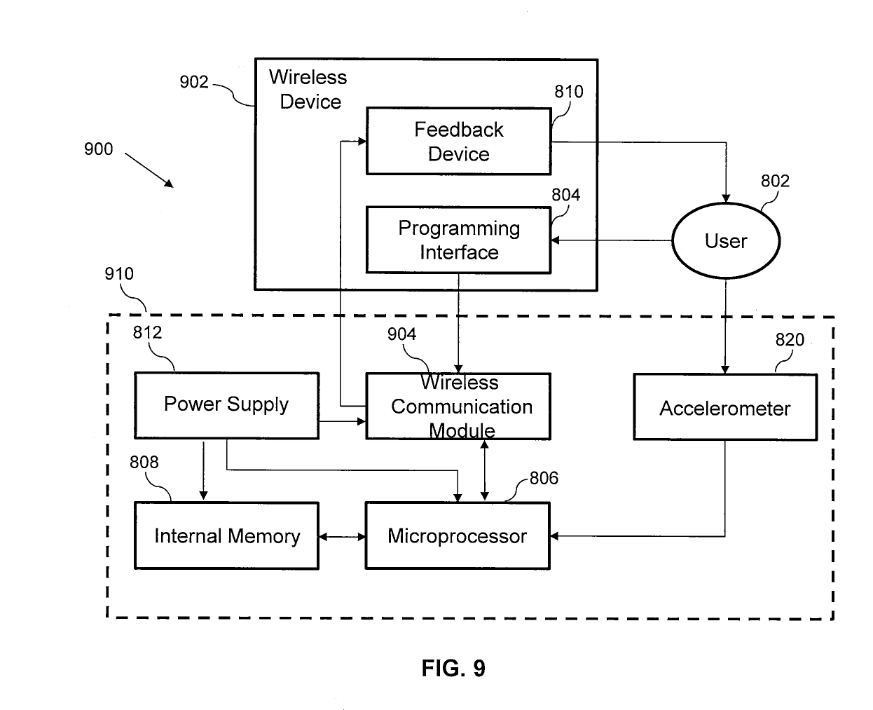

A system diagram of the operable connections between the components of a user angle monitoring system in accordance with some embodiments is shown in FIG. 9. This embodiment differs from that presented in FIG. 8 by the replacement of the feedback device 810 into a wireless device 902 together, optionally, with the programming interface 804, and the introduction of the wireless communications module 904 in the sensor unit 910. In some embodiments, the wireless device 902 may be a smart phone or smart watch used to receive user inputs from the user 802 and provide detailed feedbacks (in forms of audible, visual, and/or haptic feedbacks) to the user 802 on its screen. For example, an iPhone®, Apple Watch® or Android Smartwatch® may be used. The user 802 may also view data, including, but not limited to, current angle values, historical values and statistics and battery levels. The user 802 may make adjustments including, but not limited to, alarm settings, volume settings, feedback types, and all pre-configured values stored in the internal memory 808. The user may also receive feedback from the system via the wireless device 902, such as feedback representing an angle percentage based on the user angle of the user 802 on the seat in real-time. In the embodiment shown in FIG. 9, the feedback device 810 and the user interface 804 are operatively coupled to each other and located at or in the wireless device 902. In some embodiments, the microprocessor 806 and the internal memory 808 may also be operatively coupled to or located at or in the wireless device 902.

The wireless communication module 904 enables wireless communication between the wireless device 902 and the microprocessor 806. The microprocessor 806 receives the user's presets from the wireless communication module 904 and stores it in the internal memory 808, and receives angle signals from the accelerometer 820. After calculation of the angle percentage and comparison with thresholds, feedback signals are communicated back to the wireless device 902 via the wireless communication module 904.

In some embodiments, the pressure monitoring system 100 and the user angle monitoring system 600 can be combined such that both the pressure applied by the user and the user angle are measured and monitored. The user's behavior is estimated by the system based on pressure percentage and angle percentage. Feedback is provided based on both the pressure percentage and the angle percentage. In one example, one type of feedback (e.g. audible) is provided based on the pressure percentage, while another type of feedback (e.g. visual) is provided based on the angle percentage.

In one embodiment, the user can select one of the two measurements for the system to provide feedback. In one embodiment, the system may perform calibration of a user angle percentage based on predetermined pressure percentages; or perform calibration of a pressure percentage based on predetermined user angle percentages. For example, the system can determine a 4-degree user angle change is equivalent to a 50% pressure percentage change. A table or mapping between user angle (or user angle percentage) and pressure (or pressure percentage) may be established by the system for calibration and feedback generation.

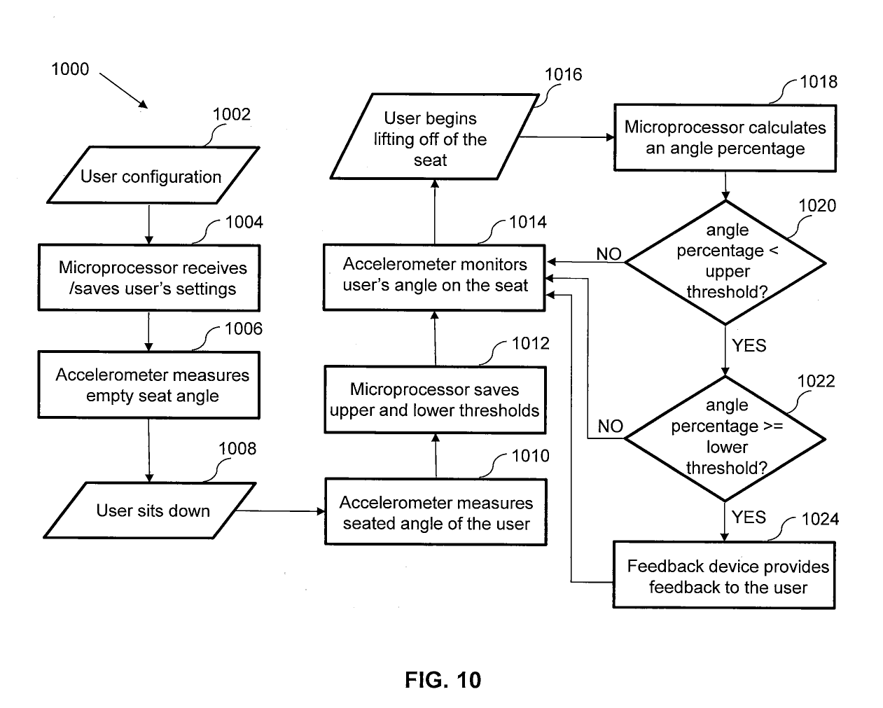

FIG. 10 is a flow diagram illustrating operations 1000 of a user angle monitoring system, e.g. the user angle monitoring system 600 in FIGS. 6-9, in accordance with some embodiments of the present disclosure. At block 1002, the user chooses the system settings using the programming interface 804. The user may choose from settings such as: feedback method, which allows the user to choose the type feedback he or she will receive, e.g. visual, auditory, haptic, or combinations thereof; feedback controls, which allows the user to control feedback options such as volume, brightness, and/or haptic type; and threshold adjustments, which allow the user to set the angle thresholds. As a non-limiting example, the “Upper Threshold”, e.g. at 90% of the user's full seated angle, represents an Angle Percentage at which the system begins to provide user feedback when the user lifts from the seat; the “Lower Threshold”, e.g. at 10% of the user's full seated angle, is a point where the feedback turns off after the user leaves the seat.

At block 1004, the settings are received by the microprocessor 806 and stored in the internal memory 808. At block 1006, the accelerometer 820 measures the “Empty Seat Angle” when the chair is not occupied by the user and/or when a force or pressure applied by the user on the seat reaches a value at which the seat is not occupied by the user. This value is calibrated to represent 0% angle percentage. At block 1008, the user sits down on the seat. At block 1010, the accelerometer measures the “Seated Angle” of the user upon sitting. This value is calibrated to represent a 100% angle percentage. The microprocessor stores the upper and lower threshold values into the internal memory 808 at block 1012. At block 1014, the accelerometer begins monitoring any changes in user angle of the user on the seat. The user begins to lift from his or her seat at block 1016.

The microprocessor calculates an angle percentage at block 1018, e.g. by reading the value from the accelerometer and dividing it by the “Seated Angle” to calculate the “Angle Percentage.” At block 1020, it is determined whether the Angle Percentage is smaller than the “Upper Threshold.” If so, the process goes to block 1022; otherwise, the process returns to block 1014 to continue monitoring the user's angle on the seat.

At block 1022, it is determined whether the Angle Percentage is larger than or equal to the “Lower Threshold.” If so, the process goes to block 1024; otherwise, the process returns to block 1014 to continue monitoring the user's angle on the seat. At block 1024, the feedback device provides auditory, visual, or haptic feedback to the user. The process continues to block 1014 to monitor the user's angle on the seat. That is, the feedback may be continuously provided until the angle percentage falls outside the range between the lower threshold and the upper threshold.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a computer readable medium. The computer readable medium can be a machine-readable storage device, a machine-readable storage substrate, a memory device, or a combination of one or more of them.

The term “processor” encompasses all apparatus, devices, and machines for processing data, including by way of example a programmable processor, a computer, or multiple processors or computers. The processor can include, in addition to hardware, code that creates an execution environment for the computer program in question, e.g., code that constitutes processor firmware, a protocol stack, a database management system, an operating system, or a combination of one or more of them.

A computer program (also known as a program, software, software application, app, script, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network or as an app on a mobile device such as a tablet, Phablet, PDA, smartphone, or wearable technology

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer or mobile device, smartphone or tablet. Generally, a processor will receive instructions and data from a read only memory or a random access memory or both. The essential elements of a computer are a processor for performing instructions and one or more data memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a mobile smartphone, a personal digital assistant (PDA), tablet, a mobile audio or video player, a game console, a Global Positioning System (GPS) receiver, to name just a few.

Computer readable media suitable for storing computer program instructions and data include all forms data memory including non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube), LCD (liquid crystal display) monitor or other monitor, for displaying information to the user and a keyboard and a pointing device, e.g., a mouse or a trackball, by which the user can provide input to the computer. Other kinds of devices can be used to provide for interaction with a user as well; for example, input from the user can be received in any form, including acoustic, speech, or tactile input.

Although examples are illustrated and described herein, embodiments are nevertheless not limited to the details shown, since various modifications and structural changes may be made therein by those of ordinary skill within the scope and range of equivalents of the claims.

What is claimed is:

1. A system comprising:
at least one sensor configured for measuring force or pressure applied by a user on a seat;
a feedback device configured for providing feedback to the user based on a force or pressure measurement from the at least one sensor;
a memory storing pre-configured values; and
a processor configured to:
receive the force or pressure measurement measured by the at least one sensor,
calculate a percentage based on the force or pressure measurement and the pre-configured values stored in the memory,
determine whether the percentage falls within a predetermined range, and
cause the feedback device to provide a feedback to the user when the calculated percentage falls within the predetermined range, wherein the feedback indicates the force or pressure applied by the user on the seat.

2. The system of claim 1, wherein the pre-configured values comprise at least one of:
a full seat value representing a force or pressure measured when the user fully sits on the seat;
an empty seat value representing a force or pressure measured when the seat is not occupied by the user;
an upper threshold; and
a lower threshold.

3. The system of claim 2, wherein the processor is further configured to:
calibrate the empty seat value to zero;
calibrate the full seat value to generate a calibrated full seat value in accordance with the calibration of the empty seat value; and
calibrate the force or pressure measured by the at least one sensor to generate a calibrated value in accordance with the calibration of the empty seat value, wherein the percentage is calculated based on a ratio between the calibrated value and the calibrated full seat value.

4. The system of claim 3, wherein:
the upper threshold represents a first percentage of the calibrated full seat value;
the lower threshold represents a second percentage of the calibrated full seat value, wherein the second percentage is smaller than the first percentage; and
the predetermined range is a range between the lower threshold and the upper threshold.

5. The system of claim 4, wherein:
the at least one sensor is configured to monitor force or pressure applied by the user on the seat; and
the processor is further configured to monitor a change of the percentage based on the monitored force or pressure from the at least one sensor.

6. The system of claim 5, in an event the percentage changes from above the upper threshold to below the upper threshold, the processor is further configured to:
determine that the user begins to lift from the seat;
cause the feedback device to start providing the feedback to the user;
compare the percentage with the lower threshold; and
cause the feedback device to stop providing the feedback to the user in an event the percentage is smaller than the lower threshold.

7. The system of claim 5, in an event the percentage changes from below the lower threshold to above the lower threshold, the processor is further configured to:
determine that the user begins to sit onto the seat;
cause the feedback device to start providing the feedback to the user;
compare the percentage with the upper threshold; and
cause the feedback device to stop providing the feedback to the user in an event the percentage is greater than the upper threshold.

8. The system of claim 1, wherein the feedback comprises at least one of: a visual feedback, an auditory feedback, a haptic feedback, and combinations thereof.

9. The system of claim 8, further comprises a user interface configured for receiving user inputs indicating at least one of the following information:
a brightness and/or color setting related to the visual feedback;
a volume and/or pitch setting related to the auditory feedback;
a haptic type setting related to the haptic feedback; and
a type of the feedback the user chooses to receive.

10. The system of claim 9, wherein the user interface is coupled to the feedback device and is further configured for receiving user inputs from the user regarding the pre-configured values during a calibration of the system for the user.

11. The system of claim 1, further comprising an accelerometer configured for measuring an angle between a thigh of the user and a ground surface under the seat, wherein the feedback device is further configured for providing feedbacks to the user based on angle measurements from the accelerometer.

12. The system of claim 1, wherein:
the seat is a wheelchair;
the at least one sensor is operatively coupled to a mat placed between a seat cushion and the wheelchair;
the feedback device is operatively coupled to the seat or is a portable device carried by the user; and
the memory and the processor are operatively coupled to the at least one sensor or coupled to the feedback device.

13. A system comprising:
at least one sensor coupled to a thigh of a user and configured for measuring an angle between the thigh of the user and a ground surface under a seat of the user;
a feedback device configured for providing feedback to the user based on an angle measurement from the at least one sensor;
a memory storing pre-configured values; and
a processor configured to:
receive the angle measurement measured by the at least one sensor,
calculate a percentage based on the angle measurement and the pre-configured values stored in the memory,
determine whether the percentage falls within a predetermined range, and
cause the feedback device to provide a feedback to the user when the calculated percentage falls within the predetermined range, wherein the feedback indicates the angle between the thigh of the user and the ground surface.

14. The system of claim 13, wherein the pre-configured values comprise at least one of:
a full seat value representing an angle measured when the user fully sits on the seat;
an empty seat value representing an angle measured when a force or pressure applied by the user on the seat reaches a value at which the seat is not occupied by the user;
an upper threshold; and
a lower threshold.

15. The system of claim 14, wherein the processor is further configured to:
calibrate the empty seat value to zero;
calibrate the full seat value to generate a calibrated full seat value in accordance with the calibration of the empty seat value; and
calibrate the angle measured by the at least one sensor to generate a calibrated value in accordance with the calibration of the empty seat value, wherein the percentage is calculated based on a ratio between the calibrated value and the calibrated full seat value.

16. The system of claim 15, wherein:
the upper threshold represents a first percentage of the calibrated full seat value;
the lower threshold represents a second percentage of the calibrated full seat value, wherein the second percentage is smaller than the first percentage; and
the predetermined range is a range between the lower threshold and the upper threshold.

17. The system of claim 16, wherein:
the at least one sensor is configured to monitor the angle between the thigh of the user and the ground surface; and
the processor is further configured to monitor a change of the percentage based on the monitored angle from the at least one sensor.

18. The system of claim 17, in an event the percentage changes from above the upper threshold to below the upper threshold, the processor is further configured to:
determine that the user begins to lift from the seat;
cause the feedback device to start providing the feedback to the user;
compare the percentage with the lower threshold; and
cause the feedback device to stop providing the feedback to the user in an event the percentage is smaller than the lower threshold.

19. The system of claim 17, in an event the percentage changes from below the lower threshold to above the lower threshold, the processor is further configured to:
determine that the user begins to sit onto the seat;
cause the feedback device to start providing the feedback to the user;
compare the percentage with the upper threshold; and
cause the feedback device to stop providing the feedback to the user in an event the percentage is greater than the upper threshold.

20. The system of claim 13, wherein the feedback comprises at least one of: a visual feedback, an auditory feedback, a haptic feedback, and combinations thereof

21. The system of claim 20, further comprises a user interface configured for receiving user inputs indicating at least one of the following information:
a brightness and/or color setting related to the visual feedback;
a volume and/or pitch setting related to the auditory feedback;
a haptic type setting related to the haptic feedback; and
a type of the feedback the user chooses to receive.

22. The system of claim 21, wherein the user interface is coupled to the feedback device and is further configured for receiving user inputs from the user regarding the pre-configured values during a calibration of the system for the user.

23. The system of claim 13, further comprising an additional sensor coupled to a mat placed between a seat cushion and the seat, wherein the additional sensor is configured for measuring force or pressure applied by the user on the seat, and wherein the feedback device is further configured for providing feedbacks to the user based on force or pressure measurements from the additional sensor.

24. The system of claim 13, wherein:
the seat is a wheelchair;
the user has impaired sensation;
the feedback device is coupled to the seat or is a portable device carried by the user; and
the memory and the processor are coupled to the at least one sensor or coupled to the feedback device.

25. A method comprising:
with at least one sensor; measuring force or pressure applied by a user on a seat;
calculating a percentage based on the force or pressure and a pre-configured value stored in a memory;
determining whether the percentage falls within a predetermined range; and
providing a feedback to the user in an event the percentage falls within the predetermined range, wherein the feedback indicates the force or pressure applied by the user on the seat.

26. The method of claim 25, further comprising:
calibrating an empty seat value of the pre-configured values to zero, wherein the empty seat value represents a force or pressure measured when the seat is not occupied by the user;
calibrating a full seat value of the pre-configured values to generate a calibrated full seat value in accordance with the calibration of the empty seat value, wherein the full seat value represents a force or pressure measured when the user fully sits on the seat;
calibrating the force or pressure measured by the at least one sensor to generate a calibrated value in accordance with the calibration of the empty seat value, wherein the percentage is calculated based on a ratio between the calibrated value and the calibrated full seat value;
monitoring force or pressure applied by the user on the seat; and
monitoring a change of the percentage based on the monitored force or pressure from the at least one sensor, wherein the pre-configured values comprise at least one of:
an upper threshold representing a first percentage of the calibrated full seat value, and
a lower threshold representing a second percentage of the calibrated full seat value, wherein the second percentage is smaller than the first percentage, and wherein the predetermined range is a range between the lower threshold and the upper threshold.

27. The method of claim 26, in an event the percentage changes from above the upper threshold to below the upper threshold, further comprising:
determining that the user begins to lift from the seat;
starting providing the feedback to the user;
comparing the percentage with the lower threshold; and
stopping providing the feedback to the user in an event the percentage is smaller than the lower threshold.

28. The method of claim 26, in an event the percentage changes from below the lower threshold to above the lower threshold, further comprising:
determining that the user begins to sit onto the seat;
starting providing the feedback to the user;
comparing the percentage with the upper threshold; and
stopping providing the feedback to the user in an event the percentage is greater than the upper threshold.

29. A system comprising:
a cushion for a seat;
at least one sensor operatively coupled to the cushion and configured for measuring force or pressure applied by a user on the seat;
a feedback device configured for providing feedback to the user based on a force or pressure measurement from the at least one sensor;
a memory storing pre-configured values; and
a processor configured to:
receive the force or pressure measurement measured by the at least one sensor,
calculate a percentage based on the force or pressure measurement and the pre-configured values stored in the memory,
determine whether the percentage falls within a predetermined range, and
cause the feedback device to provide a feedback to the user when the calculated percentage falls within the predetermined range, wherein the feedback indicates the force or pressure applied by the user on the seat.

30. The system of claim 29, further comprising the seat.
